(19) United States
(12) Patent Application Publication
(10) Pub. No.: US 2015/0073453 A1
(43) Pub. Date: Mar. 12, 2015

(54) MEDICAL DEVICE

(71) Applicant: SEIKO EPSON CORPORATION, Tokyo (JP)

(72) Inventors: Hideki KOJIMA, Matsumoto-shi (JP); Kazuaki UCHIDA, Fujimi-machi (JP); Takeshi SETO, Chofu-shi (JP); Hirokazu SEKINO, Chino-shi (JP)

(21) Appl. No.: 14/483,387

(22) Filed: Sep. 11, 2014

(30) Foreign Application Priority Data
Sep. 11, 2013 (JP) 2013-188263

(51) Int. Cl. A61B 17/3203

(52) U.S. Cl. CPC A61B 17/3203
USPC 606/167

(57) ABSTRACT

A medical device that ejects a fluid includes a fluid ejection portion having an ejection tube having an opening for ejecting the fluid and a pulsation generation portion that generates pulsation to the fluid. A control portion controls a pulsation frequency by controlling the pulsation generation portion. A measurement portion measures movement velocity of the fluid ejection portion. A receiving portion receives instructions for setting a specific frequency and a specific movement velocity from a user. A setting portion sets the specific frequency and movement velocity based on the instruction from a user. A calculation portion calculates a control constant using the specific frequency and movement velocity. The control portion controls the frequency in accordance with the movement velocity so that a value calculated by the same method as calculation of the control constant using the frequency and movement velocity falls within a predetermined range including the control constant.

This application claims the benefit of Japanese Patent Application No. 2013-188263, filed on Sep. 11, 2013. The content of the aforementioned application is incorporated herein by reference in its entirety.

### BACKGROUND

1. Technical Field

The present invention relates to a medical device.

2. Related Art

Hitherto, as a technique relating to a medical device that ejects a fluid, for example, a technique disclosed in JP-A-2008-82202 has been known. JP-A-2008-82202 discloses a medical device in which pulsation is given to a fluid by driving a piezoelectric element, and an affected part is incised or excised by ejecting a liquid to which pulsation is given onto the affected part.

However, in the medical device disclosed in JP-A-2008-82202, there is a desire for a user's convenience to be attempted to be further improved. Besides, in the medical device of the related art, a reduction in the size of, a reduction in the cost of, the resource saving of, the manufacturing facilitation of, an improvement in the usability of the device, and the like have been required.

### SUMMARY

An advantage of some aspects of the invention is to solve at least a part of the problems described above, and the invention can be implemented as the following forms.

(1) An aspect of the invention provides a medical device that ejects a fluid. The medical device includes: a fluid ejection portion that includes an ejection tube having an opening for ejecting the fluid and a pulsation generation portion, communicating with the ejection tube, which generates pulsation to the fluid; a control portion that controls a frequency of the pulsation by controlling the pulsation generation portion; a measurement portion that measures a movement velocity of the fluid ejection portion; a receiving portion that receives an instruction for setting a specific frequency and a specific movement velocity from a user; a setting portion that sets the specific frequency and the specific movement velocity on the basis of the instruction from a user; and a calculation portion that calculates a control constant using the specific frequency and the specific movement velocity. The control portion controls the frequency in accordance with the movement velocity so that a value which is calculated by the same method as a method of calculating the control constant using the frequency and the movement velocity falls within a predetermined range including the control constant.

A force for excising an object has a correlation with the number of pulsating flows ejected per unit length of the object. According to such an aspect, control is performed so that the number of pulsating flows ejected per unit length of the object comes close to the number which is set by an instruction from a user, thereby it is possible to maintain an excision force which is set by the instruction from a user.

(2) In the medical device described above, the setting portion may set the frequency at a timing when the setting instruction is received, to the specific frequency, and may set the movement velocity at a timing when the setting instruction is received, to the specific movement velocity.

According to this configuration, when the setting portion receives the setting instruction at a timing when a user's favorite excision force is realized, it is possible to maintain a user's favorite excision force.

(3) In the medical device described above, the calculation portion may calculate the control constant by dividing the specific frequency by the specific movement velocity, and the control portion may control the frequency so that a value of the frequency comes close to a value obtained by multiplying the control constant and the movement velocity together.

According to this configuration, it is possible to calculate the number of pulsating flows ejected per unit length of the object as the control constant, and to easily control the frequency.

(4) In the medical device described above, the control portion may control the frequency so that when the movement velocity is larger than a predetermined upper limit threshold, the frequency has a value smaller than a value of the frequency in a case where the frequency is controlled using the control constant.

According to this configuration, when the movement velocity becomes greater than the predetermined upper limit threshold, the frequency has a small value. Therefore, for example, when the movement velocity becomes greater against a user's intention, it is possible to reduce an excision force.

(5) In the medical device described above, the control portion may control the frequency so that when the movement velocity is less than a predetermined lower limit threshold, the frequency has a value smaller than a value of the frequency in a case where the frequency is controlled using the control constant.

According to this configuration, when the movement velocity is less than the predetermined lower limit threshold, the frequency has a small value. Therefore, for example, when the apical end of the fluid ejection tube continues to remain at the same position of the object which is undesired against a user's intention, or when a user interrupts the excision of the object or the like and thus the movement velocity becomes lower, it is possible to reduce an excision force.

(6) In the medical device described above, the control portion may perform control so that an output of the pulsation generation portion is reduced when the movement velocity is larger than a predetermined upper limit threshold.

According to this configuration, when the movement velocity becomes greater than the predetermined upper limit threshold, the output of the pulsation generation portion is reduced. Therefore, for example, when the movement velocity becomes greater against a user's intention, it is possible to reduce an excision force.

(7) In the medical device described above, the control portion may perform control so that an output of the pulsation generation portion is reduced when the movement velocity is less than a predetermined lower limit threshold.

According to this configuration, when the movement velocity is less than the predetermined lower limit threshold, the output of the pulsation generation portion is reduced. Therefore, for example, when the apical end of the fluid ejection tube continues to remain at the same position of the object which is undesired against a user's intention, or when a user interrupts the excision of the object and thus the movement velocity becomes lower, it is possible to reduce the excision force.

Not all of a plurality of components included in the respective aspects of the invention described above are essential. In order to solve some or all of the aforementioned problems, or to achieve some or all of the effects described in this specification, regarding some components of the plurality of components, it is possible to appropriately perform change, deletion, replacement with other new components, and deletion of a portion of limited contents. Further, in order to solve some or all of the aforementioned problems, or to achieve some or all of the effects described in this specification, some or all of the technical features included in an aspect of the invention described above can also be combined with some or all of the technical features included in other aspects of the invention, to thereby form an independent aspect of the invention.

For example, an aspect of the invention can be realized as device including one or more elements within six elements of the fluid ejection portion, the control portion, the measurement portion, the receiving portion, the setting portion, and the calculation portion. That is, this device may or may not include the fluid ejection portion. In addition, the device may or may not include the control portion. In addition, the device may or may not include the measurement portion. In addition, the device may or may not include the receiving portion. In addition, the device may or may not include the setting portion. In addition, the device may or may not include the calculation portion. The fluid ejection portion may be configured as, for example, a fluid ejection portion provided with an ejection tube having an opening for ejecting the fluid and a pulsation generation portion, communicating with the ejection tube, which generates pulsation in the fluid. The control portion maybe configured as, for example, a control portion that controls a frequency of the pulsation by controlling the pulsation generation portion. The measurement portion may be configured as, for example, a measurement portion that measures a movement velocity of the opening. The receiving portion may be configured as, for example, a receiving portion that receives an instruction for setting a specific frequency and a specific movement velocity from a user. The setting portion may be configured as, for example, a setting portion that sets the specific frequency and the specific movement velocity on the basis of the instruction from a user. The calculation portion may be configured as, for example, a calculation portion that calculates a control constant using the specific frequency and the specific movement velocity. In addition, the control portion may be configured as, for example, a control portion that controls the frequency in accordance with the movement velocity so that a value which is calculated by the same method as a method of calculating the control constant using the frequency and the movement velocity falls within a predetermined range including the control constant. Such a device can be realized, for example, as a medical device that ejects a fluid, but can be realized by devices other than the medical device that ejects a fluid. According to such an aspect, it is possible to solve at least one of various problems of a reduction in the size of, a reduction in the cost of, the resource saving of, the manufacturing facilitation of, an improvement in the usability of a device, and the like. Some or all of technical features of each aspect of the medical device that ejects a fluid stated above can be applied to this device entirely.

The invention can also be implemented as various forms other than the device. For example, the invention can be implemented as forms such as a method of manufacturing a medical device that ejects a fluid, a method of controlling a medical device that ejects a fluid, a computer program for realizing the control method, a non-transitory recording medium having the computer program recorded thereon, and the like.

### BRIEF DESCRIPTION OF THE DRAWINGS

The invention will be described with reference to the accompanying drawings, wherein like numbers reference like elements.

FIG. 1 is a diagram illustrating a configuration of a medical device as an embodiment of the invention.

FIG. 2 is an enlarged cross-sectional view illustrating a portion of an internal configuration of a handpiece.

FIG. 3 is a diagram illustrating an example of a waveform of a drive voltage which is applied to a piezoelectric element.

FIG. 4 is a diagram illustrating a correspondence relation between the waveform of the drive voltage and a modified state of a diaphragm.

FIG. 5 is a diagram illustrating a relationship between a drive frequency F [Hz] of the drive voltage which is applied the piezoelectric element and a depth [mm] where an affected part is excised, in a graph form.

FIG. 6 is a diagram illustrating a relationship between a movement velocity V [mm/s] of a fluid ejection tube and the drive frequency F [Hz] of the drive voltage which is applied to the piezoelectric element.

FIG. 7 is a diagram illustrating a control pattern in a medical device as a second embodiment.

FIG. 8 is a diagram illustrating a control pattern in a medical device as a third embodiment.

FIG. 9 is a diagram illustrating a control pattern in a medical device as a fourth embodiment.

FIG. 10 is a diagram illustrating a control pattern in a medical device as a fifth embodiment.

### DESCRIPTION OF EXEMPLARY EMBODIMENTS

Next, embodiments of the invention will be described in the following order on the basis of the embodiments.

A. First Embodiment

B. Second Embodiment

C. Third Embodiment

D. Fourth Embodiment

E. Fifth Embodiment

F. Modification Example

### A. FIRST EMBODIMENT

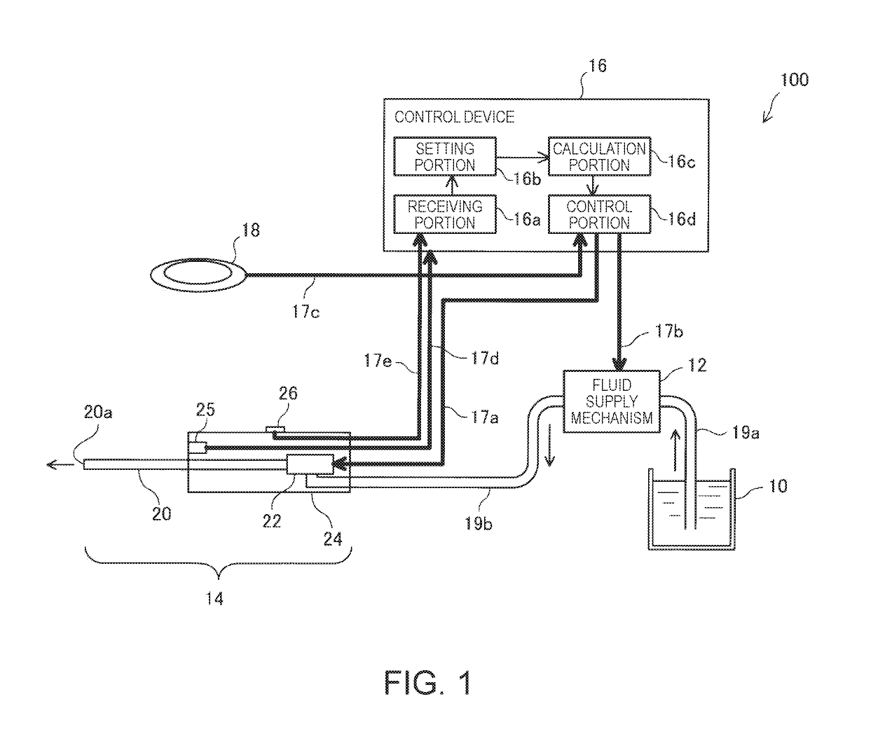

FIG. 1 is a diagram illustrating a configuration of a medical device 100 as an embodiment of the invention. The medical device 100 has a function as a scalpel for performing medical treatment such as incision or excision of an affected part of a patient by ejecting a fluid onto a biological tissue which is an affected part.

The medical device 100 includes a fluid container 10, a fluid supply mechanism 12, a handpiece 14, a control device 16, and an ejection switch 18. The fluid container 10 and the fluid supply mechanism 12 are connected to each other by a connection tube 19a, and the fluid supply mechanism 12 and the handpiece 14 are connected to each other by a connection tube 19b. In the present embodiment, the connection tubes 19a and 19b are formed of a resin.

The fluid container 10 accommodates a physiological saline solution as a fluid which is supplied to the handpiece 14. Here, the fluid container 10 may accommodate other fluids, such as, for example, pure water or a drug solution which are not harmful even during ejection onto a biological tissue, instead of the physiological saline solution.

The fluid supply mechanism 12 supplies a fluid accommodated in the fluid container 10 to the handpiece 14 through the connection tubes 19a and 19b. In the present embodiment, a pump is used as the fluid supply mechanism 12.

The handpiece 14 is an appliance which is manipulated by an operator with the handpiece held in his/her hand, and includes a fluid ejection tube 20, a pulsation generation portion 22, and a housing 24. When a drive voltage is applied through a voltage application cable 17a, the pulsation generation portion 22 gives pulsation to a fluid which is supplied through the connection tube 19b. The fluid to which pulsation is given is ejected from an opening 20a of an apical end of the fluid ejection tube 20 at high speed. An operator incises, excises or the like, for example, an affected part by applying a fluid, ejected from the handpiece 14, to which pulsation is given, to a biological tissue which is an affected part of a patient. Meanwhile, hereinafter, a fluid to which pulsation is given is also called a pulsating flow or a pulse flow. Meanwhile, the magnitude (volume ejected by one-time driving) and the intensity of a pulsating flow is changed when the magnitude of a drive voltage is changed, and the frequency of occurrence of the pulsating flow is changed when the drive frequency of a drive voltage is changed.

The control device 16 controls a drive voltage which is applied to the pulsation generation portion 22 through the voltage application cable 17a, and controls the start and stop of the fluid supply mechanism 12 through a control cable 17b.

The ejection switch 18 is a switch which is manipulated by an operator, and is connected to the control device 16 through a control cable 17c. In the present embodiment, the ejection switch 18 is a foot switch which is manipulated by an operator using his/her foot.

When the ejection switch 18 is turned on by an operator, the control device 16 instructs the fluid supply mechanism 12 to start a supply of a fluid, and applies a drive voltage to the pulsation generation portion 22. A fluid to which pulsation is given is ejected at high speed from the opening 20a of the apical end of the fluid ejection tube 20 of the handpiece 14.

In the present embodiment, the handpiece 14 further includes an acceleration sensor 25 and an information acquisition switch 26. The acceleration sensor 25 is provided in the vicinity of the apical end of the housing 24, and detects acceleration. In the present embodiment, the acceleration sensor 25 is a semiconductor-type 3-axis acceleration sensor. The detected acceleration is supplied to the control device 16 through a control cable 17d. the information acquisition switch 26 is a switch which is depressed by an operator. When the information acquisition switch 26 is depressed by an operator, a signal indicating that the information acquisition switch 26 is depressed is supplied to the control device 16 through a control cable 17e.

The control device 16 calculates the movement velocity V of the fluid ejection tube 20 on the basis of the acceleration detected by the acceleration sensor 25 and a positional relation between the acceleration sensor 25 and the fluid ejection tube 20. In addition, the control device 16 sets the movement velocity V of the fluid ejection tube 20 at a timing when the information acquisition switch 26 is depressed, as a specific movement velocity Vs, and sets the drive frequency F of a drive voltage which is applied to the pulsation generation portion 22 at the timing, as a specific drive frequency Fs. The reason for setting the specific movement velocity Vs and the specific drive frequency Fs will be described later. In addition, the control device 16 also functions as a receiving portion 16a, a setting portion 16b, a calculation portion 16c, and a control portion 16d which are described later.

FIG. 2 is an enlarged cross-sectional view illustrating a portion of the internal configuration of the handpiece 14. The pulsation generation portion 22 that gives pulsation to a fluid supplied from the fluid supply mechanism 12 is provided in the inside of the housing 24 of the handpiece 14. The pulsation generation portion 22 includes a piezoelectric element 30, a diaphragm 32, a first case 34, a second case 36, and a third case 38.

In the pulsation generation portion 22, an inlet channel 40, a fluid chamber 42, and an outlet channel 44 are formed as a channel through which a fluid supplied from the fluid supply mechanism 12 passes. In the present embodiment, the inlet channel 40 and the outlet channel 44 are formed in the first case 34, and the fluid chamber 42 is formed between the first case 34 and the diaphragm 32. The connection tube 19b is connected to the inlet channel 40, and the fluid ejection tube 20 is connected to the outlet channel 44.

The diaphragm 32 is a disk-shaped metal sheet, and the outer circumference portion thereof is interposed between the first case 34 and the second case 36 and is fixed thereto.

The piezoelectric element 30 is an actuator which is operated by a drive voltage applied from the control device 16. The piezoelectric element 30 changes the volume of the fluid chamber 42 formed between the diaphragm 32 and the first case 34, to thereby change the pressure of a fluid within the fluid chamber 42. In the present embodiment, the piezoelectric element 30 is a laminated piezoelectric element, and is configured such that one end thereof is fixed to the diaphragm 32 and the other end thereof is fixed to the third case 38.

When a drive voltage applied to the piezoelectric element 30 becomes greater, the piezoelectric element 30 extends, and the diaphragm 32 is pushed to the piezoelectric element 30 and bends to the fluid chamber 42 side. When the diaphragm 32 bends to the fluid chamber 42 side, the volume of the fluid chamber 42 decreases, and the fluid within the fluid chamber 42 is extruded from the fluid chamber 42. In the present embodiment, the inside diameter of the outlet channel 44 is larger than the inside diameter of the inlet channel 40. That is, since the inertance of the outlet channel 44 is smaller than the inertance of the inlet channel 40, the fluid within the fluid chamber 42 is extruded from the fluid chamber 42 through the outlet channel 44.

On the other hand, when the drive voltage applied piezoelectric element 30 becomes lower, the volume of the fluid chamber 42 becomes greater due to a reduction in the size of the piezoelectric element 30, and a fluid is supplied from the inlet channel 40 into the fluid chamber 42.

Since the drive voltage applied to the piezoelectric element 30 repeats turn-on (maximum voltage) and turn-off (0 V) at a high frequency (for example, 400 Hz), the enlargement and reduction of the volume of the fluid chamber 42 are repeated, and pulsation is given to the fluid. The fluid extruded from the fluid chamber 42 is ejected from the nozzle 20a (opening 20a) of the apical end of the fluid ejection tube 20.

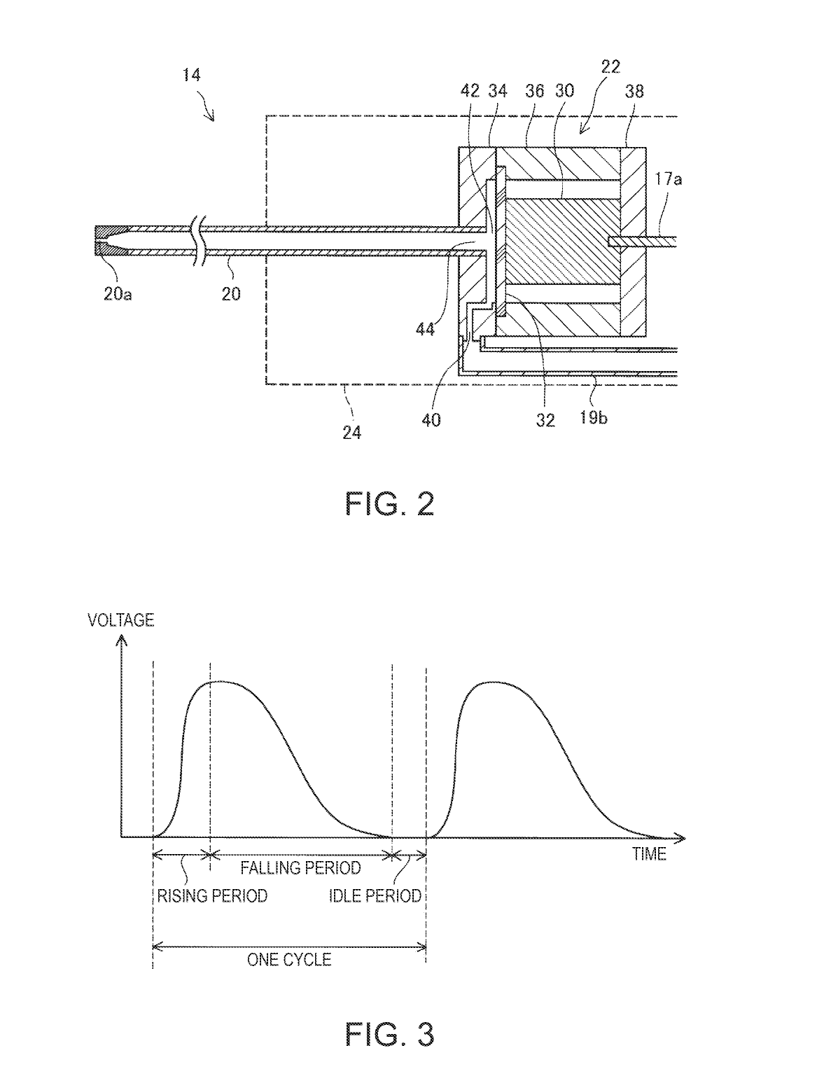

FIG. 3 is a diagram illustrating an example of a waveform of a drive voltage which is applied to the piezoelectric element 30. In FIG. 3, the horizontal axis represents a time, and the vertical axis represents a drive voltage. One cycle of the waveform of the drive voltage is composed of a rising period in which a voltage increases, a falling period in which a voltage decreases, and an idle period in which a voltage is not applied.

In the present embodiment, the waveform of the drive voltage in the rising period is offset in a positive voltage direction, and is a waveform of ½ cycle of an SIN waveform of which the phase is shifted by −90 degrees. The waveform of the drive voltage in the falling period is offset in a positive voltage direction, and is a waveform of ½ cycle of the SIN waveform of which the phase is shifted by +90 degrees. The cycle of the SIN waveform in the falling period becomes greater than the cycle of the SIN waveform in the rising period.

In the present embodiment, when the magnitude of the drive voltage is changed, the maximum value of the waveform shown in FIG. 3 is changed. In addition, when the frequency of the drive voltage is changed, the waveform in the rising period and the falling period is not changed, and the length of the idle period is changed.

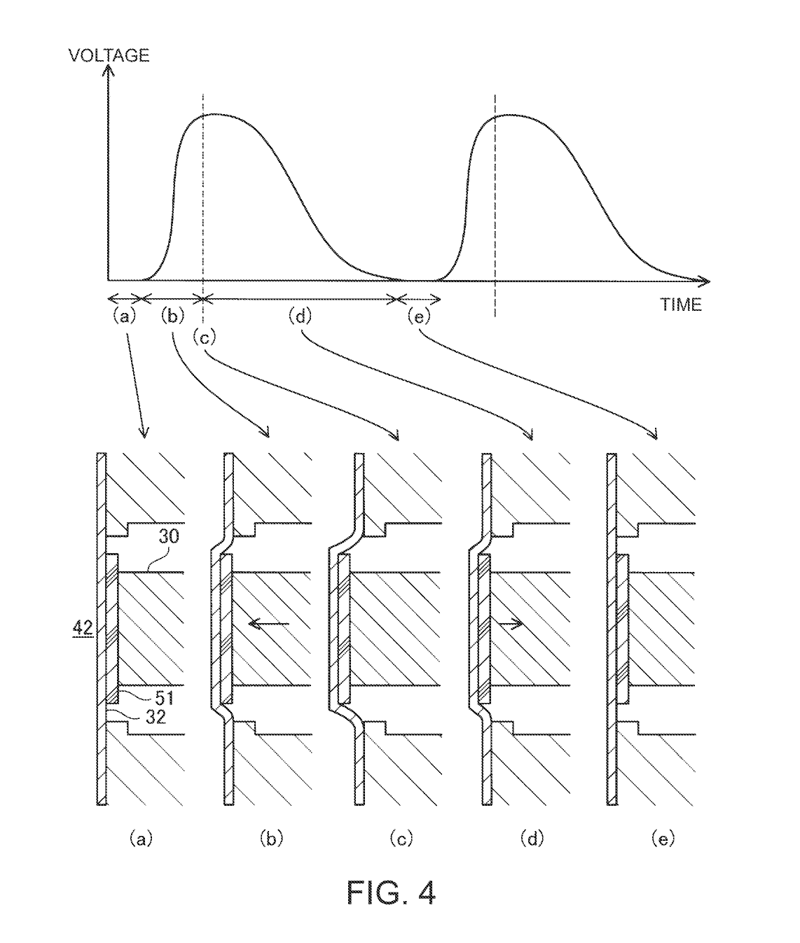

FIG. 4 is a diagram illustrating a correspondence relation between the waveform of the drive voltage and the modified state of the diaphragm 32. Meanwhile, in FIG. 4, a reinforcement member 51 is provided between the piezoelectric element 30 and the diaphragm 32. In the idle period (a), since the drive voltage is not applied, the piezoelectric element 30 does not extend, and the diaphragm 32 does not bend. In the rising period (b), since the drive voltage becomes greater, the piezoelectric element 30 extends, the diaphragm 32 bends to the fluid chamber 42 side, and the volume of the fluid chamber 42 decreases.

Since the drive voltage becomes maximum at a timing in (c) of the drawing, the length of the piezoelectric element 30 also becomes maximum, and the volume of the fluid chamber 42 becomes minimum. Since the drive voltage becomes lower in the falling period (d), the piezoelectric element 30 starts returning to its original dimensions, and the volume of the fluid chamber 42 starts returning to its original dimensions. Since the drive voltage is not applied in the idle period (e), the piezoelectric element 30 returns to its original dimensions, and the volume of the fluid chamber 42 returns to its original dimensions. A series of operations shown in (a) to (e) of the drawing are repeated, and thus the fluid within the fluid chamber 42 is extruded to the fluid ejection tube 20.

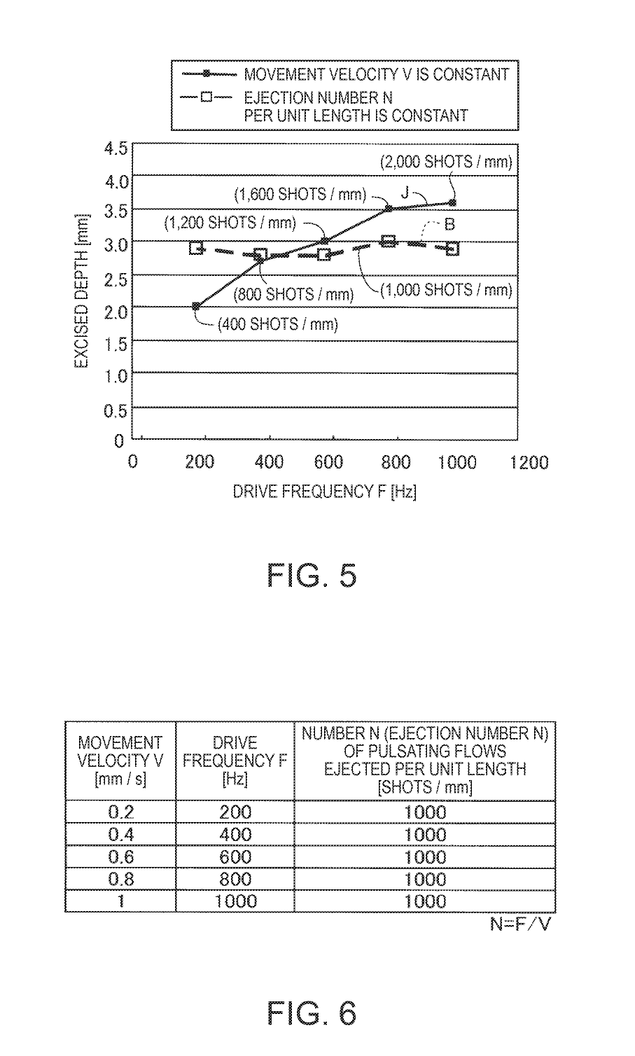

FIG. 5 is a diagram illustrating a relationship between the drive frequency F [Hz] of the drive voltage applied to the piezoelectric element 30 and the depth [mm] to which an affected part is excised, in a graph form. A solid line J shown in FIG. 5 shows a case where the fluid ejection tube 20 is moved at a constant velocity with respect to an affected part, and only the drive frequency F is changed. On the other hand, a broken line B shown in FIG. 5 shows a case where the movement velocity V and the drive frequency F of the fluid ejection tube 20 are adjusted so that the number N of pulsating flows (hereinafter, also simply called “ejection number N”) ejected per unit length of an affected part becomes constant. In the example shown in FIG. 5, the movement velocity V and the drive frequency F of the fluid ejection tube 20 are adjusted so that the ejection number N per unit length is 1,000 shots/mm.

According to FIG. 5, it can be understood that when the ejection number N per unit length increases, the excision depth increases. In addition, when the ejection numbers N per unit length are the same as each other, even when the movement velocity V and the drive frequency F of the fluid ejection tube 20 are different, it can be understood that the excision depths are substantially the same as each other. That is, it can be understood that an excision force for excising an affected part has a correlation with the ejection number N per unit length.

FIG. 6 is a diagram illustrating a relationship between the movement velocity V [mm/s] of the fluid ejection tube 20 and the drive frequency F [Hz] of the drive voltage which is applied to the piezoelectric element 30. As shown in FIG. 6, when the drive frequency F is controlled in accordance with the movement velocity V of the fluid ejection tube 20, the ejection number N per unit length becomes constant. In the present embodiment, the control portion 16d controls the drive frequency F in accordance with the measured movement velocity V so that the ejection number N per unit length falls within a predetermined range. In this manner, even when the movement velocity V of the fluid ejection tube 20 is changed, the excision force can be made substantially constant, and the depth to which the affected part is excised can be made substantially constant. In the present embodiment, the control device 16 controls the drive frequency F so as to maintain the excision force at a timing when the information acquisition switch 26 is depressed by an operator. The details of control are as follows.

The receiving portion 16a receives an instruction for setting the specific drive frequency Fs and the specific movement velocity Vs from an operator. In the present embodiment, the receiving portion 16a receives a signal indicating that the information acquisition switch 26 is depressed through the control cable 17e.

The setting portion 16b sets the specific drive frequency Fs and the specific movement velocity Vs on the basis of the instruction from an operator. In the present embodiment, the setting portion 16b sets the drive frequency F at a timing when the information acquisition switch 26 is depressed to the specific drive frequency Fs, and sets the movement velocity V of the fluid ejection tube 20 at a timing when the information acquisition switch 26 is depressed to the specific movement velocity Vs.

The calculation portion 16c calculates a control constant Ns using the set specific drive frequency Fs and the set specific movement velocity Vs. In the present embodiment, the calculation portion 16c calculates the control constant Ns by dividing the specific drive frequency Fs by the specific movement velocity Vs.

The control portion 16d controls the drive frequency F in accordance with the movement velocity V of the fluid ejection tube 20 so that a value calculated by the same method as a method of calculating the control constant Ns using the drive frequency F and the movement velocity V of the fluid ejection tube 20, that is, the ejection number N per unit length falls within a predetermined range including the control constant Ns. In the present embodiment, the control portion 16d controls the drive frequency F so that the value of the drive frequency F comes close to a value obtained by multiplying the control constant Ns and the movement velocity V of the fluid ejection tube 20 together. Therefore, even when the movement velocity V of the fluid ejection tube 20 is changed, it is possible to easily control the drive frequency F, and to maintain the excision force at a timing when the information acquisition switch 26 is depressed.

In this manner, according to the present embodiment, control is performed so that the ejection number N per unit length comes close to the number (control constant Ns) which is set by an instruction from a user. Therefore, even when the movement velocity V of the fluid ejection tube 20 is changed, it is possible to maintain the excision force which is set by an instruction from an operator.

Further, according to the present embodiment, when a user depresses the information acquisition switch 26 at a timing when a favorite excision force is realized, it is possible to maintain the excision force at a timing when the favorite excision force is realized. Meanwhile, the fluid ejection tube 20 and the pulsation generation portion 22 are equivalent to a “fluid ejection portion” according to the invention.

### B. SECOND EMBODIMENT

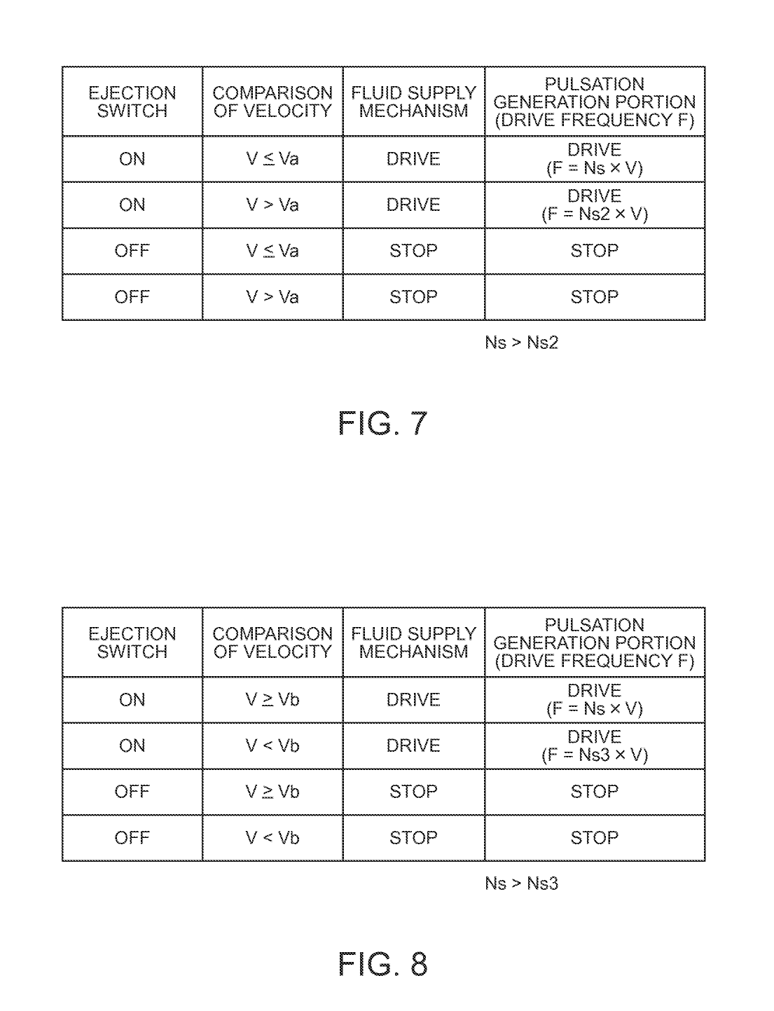

FIG. 7 is a diagram illustrating a control pattern in a medical device 100 as a second embodiment. The basic configuration of the second embodiment is the same as that of the above-mentioned first embodiment. When the ejection switch 18 is depressed by an operator and is turned on, the fluid supply mechanism 12 and the pulsation generation portion 22 are driven. On the other hand, when the ejection switch 18 is not depressed by an operator and is turned off, the fluid supply mechanism 12 and the pulsation generation portion 22 are stopped.

In the present embodiment, the control portion 16d controls the drive frequency F so that when the movement velocity V of the fluid ejection tube 20 is larger than a predetermined upper limit threshold Va, the drive frequency F has a value smaller than the value of the drive frequency F in a case where the drive frequency F is controlled using the control constant Ns. Meanwhile, the predetermined upper limit threshold Va may be set on the basis of the specific movement velocity Vs. For example, the predetermined upper limit threshold Va may be preferably a value greater than the specific movement velocity Vs, and may be preferably, for example, a value of 1.2 times the specific movement velocity Vs. A specific control method of the drive frequency F is as follows.

The calculation portion 16c calculates the control constant Ns in the same manner as that in the first embodiment, and calculates a second control constant Ns2 by multiplying the control constant Ns and a predetermined constant less than 1 together. For example, the calculation portion 16c calculates the second control constant Ns2 on the basis of the following expression.

Ns2=Ns×0.5

The control portion 16d compares the movement velocity V of the fluid ejection tube 20 with the predetermined upper limit threshold Va. When the movement velocity V of the fluid ejection tube 20 is equal to or less than the predetermined upper limit threshold Va, the control portion 16d controls the drive frequency F so that the value of the drive frequency F comes close to a value obtained by multiplying the control constant Ns and the movement velocity V of the fluid ejection tube 20 together.

On the other hand, when the movement velocity V of the fluid ejection tube 20 is greater than the predetermined upper limit threshold Va, the control portion 16d controls the drive frequency F so that the value of the drive frequency F comes close to a value obtained by multiplying the second control constant Ns2 and the movement velocity V of the fluid ejection tube 20 together.

In this manner, when the movement velocity V of the fluid ejection tube 20 is greater than the predetermined upper limit threshold Va, the ejection number N per unit length becomes smaller than the ejection number N per unit length in a case where the movement velocity V of the fluid ejection tube 20 is equal to or less than the predetermined upper limit threshold Va.

Therefore, according to the present embodiment, when the movement velocity V of the fluid ejection tube 20 becomes greater against an operator's intention, the ejection number N per unit length has a small value, and thus it is possible to reduce the excision force. For example, when the movement velocity V of the fluid ejection tube 20 becomes greater against an operator's intention, and the apical end of the fluid ejection tube 20 moves to an undesired position of an affected part, it is possible to reduce the excision force. As a result, it is possible to improve the safety of the medical device 100.

### C. THIRD EMBODIMENT

FIG. 8 is a diagram illustrating a control pattern in a medical device 100 as a third embodiment. The basic configuration of the third embodiment is the same as that of the above-mentioned first embodiment. In the present embodiment, when the control portion 16d controls the drive frequency F so that when the movement velocity V of the fluid ejection tube 20 is smaller than a predetermined lower limit threshold Vb, the drive frequency F has a value smaller than the value of the drive frequency F in a case where the drive frequency F is controlled using the control constant Ns. Meanwhile, the predetermined lower limit threshold Vb may be set on the basis of the specific movement velocity Vs. For example, the predetermined lower limit threshold Vb may be a value less than the specific movement velocity Vs, and may be, for example, a value 0.8 times the specific movement velocity Vs. A specific control method of the drive frequency F is as follows.

The calculation portion 16c calculates the control constant Ns in the same manner as that in the first embodiment, and calculates a third control constant Ns3 by multiplying the control constant Ns and a predetermined constant less than 1 together. For example, the calculation portion 16c calculates the third control constant Ns3 on the basis of the following expression.

Ns3=Ns×0.5

The control portion 16d compares the movement velocity V of the fluid ejection tube 20 with the predetermined lower limit threshold Vb. When the movement velocity V of the fluid ejection tube 20 is equal to or greater than the predetermined lower limit threshold Vb, the control portion 16d controls the drive frequency F so that the value of the drive frequency F comes close to a value obtained by multiplying the control constant Ns and the movement velocity V of the fluid ejection tube 20 together.

On the other hand, when the movement velocity V of the fluid ejection tube 20 is less than the predetermined lower limit threshold Vb, the control portion 16d controls the drive frequency F so that the value of the drive frequency F comes close to a value obtained by multiplying the third control constant Ns3 and the movement velocity V of the fluid ejection tube 20 together.

In this manner, when the movement velocity V of the fluid ejection tube 20 is less than the predetermined lower limit threshold Vb, the ejection number N per unit length becomes smaller than the ejection number N per unit length in a case where the movement velocity V of the fluid ejection tube 20 is equal to or greater than the predetermined lower limit threshold Vb.

Therefore, according to the present embodiment, when the movement velocity V of the fluid ejection tube 20 becomes lower, the ejection number N per unit length has a small value, it is possible to reduce the excision force. For example, when the apical end of the fluid ejection tube 20 continues to remain at the same position of an affected part which is undesired, against an operator's intention, or when an operator interrupts the excision of an affected part or the like and thus the movement velocity V becomes lower, it is possible to reduce the excision force. As a result, it is possible to improve the safety of the medical device 100.

### D. FOURTH EMBODIMENT

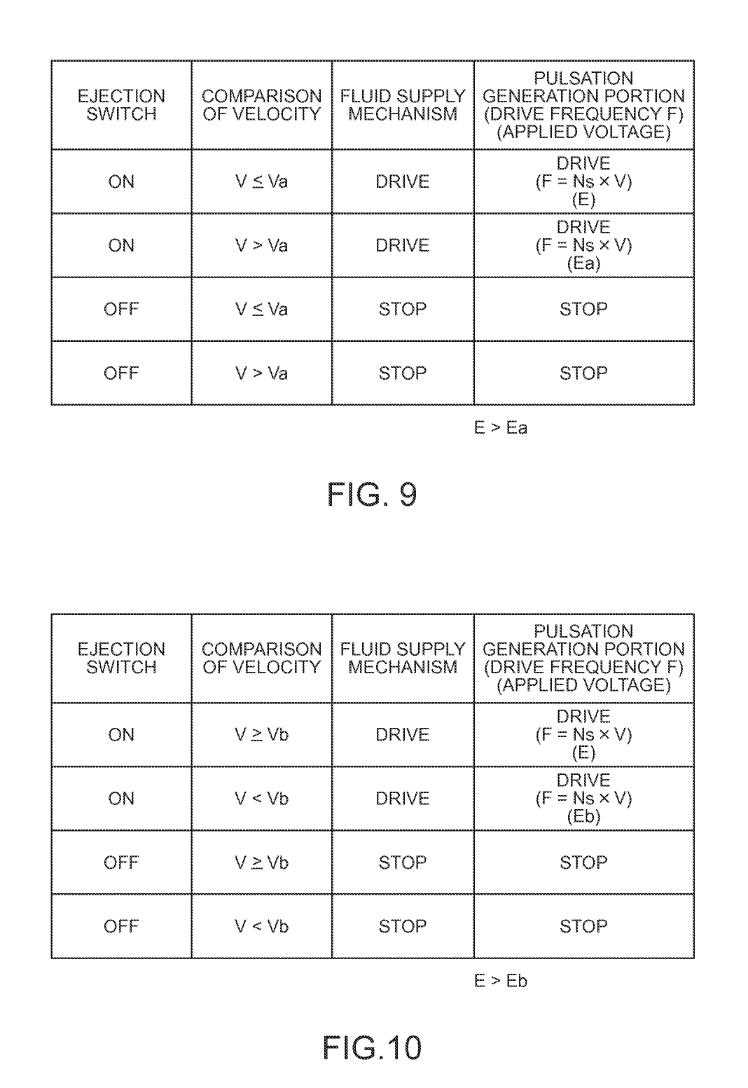

FIG. 9 is a diagram illustrating a control pattern in a medical device 100 as a fourth embodiment. The basic configuration of the fourth embodiment is the same as that of the above-mentioned first embodiment. In the present embodiment, the control portion 16d reduces the magnitude of the drive voltage which is applied to the piezoelectric element 30 when the movement velocity V of the fluid ejection tube 20 is larger than the predetermined upper limit threshold Va while the drive frequency F is controlled in the same manner as that in the above-mentioned first embodiment. Meanwhile, the predetermined upper limit threshold Va maybe set on the basis of the specific movement velocity Vs. For example, the predetermined upper limit threshold Va may be a value greater than the specific movement velocity Vs, and may be, for example, a value 1.2 times the specific movement velocity Vs. A specific control method of the drive frequency F and the drive voltage is as follows.

The control portion 16d compares the movement velocity V of the fluid ejection tube 20 with the predetermined upper limit threshold Va. When the movement velocity V of the fluid ejection tube 20 is equal to or less than the predetermined upper limit threshold Va, the control portion 16d controls the drive frequency F so that the maximum value of the drive voltage which is applied to the piezoelectric element 30 is set to a predetermined value E, and that the value of the drive frequency F comes close to a value obtained by multiplying the control constant Ns and the movement velocity V of the fluid ejection tube 20 together.

On the other hand, when the movement velocity V of the fluid ejection tube 20 is greater than the predetermined upper limit threshold Va, the control portion 16d controls the drive frequency F so that the maximum value of the drive voltage which is applied to the piezoelectric element 30 is set to a predetermined value Ea smaller than the above-mentioned predetermined value E, and that the value of the drive frequency F comes close to a value obtained by multiplying the control constant Ns and the movement velocity V of the fluid ejection tube 20 together. Meanwhile, Ea may be a value smaller than E, may be, for example, a value of 0.5 times E, and may be 0.

In this manner, when the movement velocity V of the fluid ejection tube 20 is greater than the predetermined upper limit threshold Va, the intensity of a pulsating flow which is ejected from the fluid ejection tube 20 becomes lower than the intensity of a pulsating flow in a case where the movement velocity V of the fluid ejection tube 20 is equal to or less than the predetermined upper limit threshold Va.

For example, when the movement velocity V of the fluid ejection tube 20 becomes greater against an operator's intention, and the apical end of the fluid ejection tube 20 moves to an undesired position of an affected part, it is possible to reduce the excision force. As a result, it is possible to improve the safety of the medical device 100.

### E. FIFTH EMBODIMENT

FIG. 10 is a diagram illustrating a control pattern in a medical device 100 as a fifth embodiment. The basic configuration of the fifth embodiment is the same as that of the above-mentioned first embodiment. In the present embodiment, the control portion 16d reduces the magnitude of the drive voltage which is applied to the piezoelectric element 30 when the movement velocity V of the fluid ejection tube 20 is smaller than the predetermined lower limit threshold Vb while the drive frequency F is controlled in the same manner as that in the above-mentioned first embodiment. Meanwhile, the predetermined lower limit threshold Vb may be set on the basis of the specific movement velocity Vs. For example, the predetermined lower limit threshold Vb maybe a value less than the specific movement velocity Vs, and may be, for example, a value of 0.8 times the specific movement velocity Vs. A specific control method of the drive frequency F and the drive voltage is as follows.

The control portion 16d compares the movement velocity V of the fluid ejection tube 20 with the predetermined upper limit threshold Va. When the movement velocity V of the fluid ejection tube 20 is equal to or greater than the predetermined lower limit threshold Vb, the control portion 16d controls the drive frequency F so that the maximum value of the drive voltage which is applied to the piezoelectric element 30 is set to the predetermined value E, and that the value of the drive frequency F comes close to a value obtained by multiplying the control constant Ns and the movement velocity V of the fluid ejection tube 20 together.

On the other hand, when the movement velocity V of the fluid ejection tube 20 is less than the predetermined lower limit threshold Vb, the control portion 16d controls the drive frequency F so that the maximum value of the drive voltage which is applied to the piezoelectric element 30 is set to a predetermined value Eb smaller than the above-mentioned predetermined value E, and that the value of the drive frequency F comes close to a value obtained by multiplying the control constant Ns and the movement velocity V of the fluid ejection tube 20 together. Meanwhile, Eb may be a value smaller than E, may be, for example, a value 0.5 times E, and may be 0.

In this manner, when the movement velocity V of the fluid ejection tube 20 is less than the predetermined lower limit threshold Vb, the intensity of a pulsating flow which is ejected from the fluid ejection tube 20 becomes lower than the intensity of a pulsating flow in a case where the movement velocity V of the fluid ejection tube 20 is equal to or greater than the predetermined lower limit threshold Vb.

Therefore, according to the present embodiment, when the movement velocity V of the fluid ejection tube 20 becomes lower, it is possible to reduce the excision force. For example, when the apical end of the fluid ejection tube 20 continues to remain at the same position of an affected part which is undesired, against an operator's intention, or when an operator interrupts the excision of an affected part or the like and thus the movement velocity V becomes lower, it is possible to reduce the excision force. As a result, it is possible to improve the safety of the medical device 100.

### F. MODIFICATION EXAMPLE

Meanwhile, the invention is not limited to the above-mentioned embodiments, and can be implemented in various aspects without the gist of the invention. For example, the following modifications can be made.

### Modification Example 1

In the above-mentioned embodiment, a user interface for receiving an instruction for setting the specific drive frequency Fs and the specific movement velocity Vs from an operator may be connected to the control device 16. In addition, the setting portion 16b may set the specific drive frequency Fs from the average value of the drive frequency F in the past specific period, on the basis of the instruction from an operator. In addition, the setting portion 16b may set the specific movement velocity Vs from the average value of the movement velocity V of the fluid ejection tube 20 in the past specific period, on the basis of the instruction from an operator.

### Modification Example 2

In the above-mentioned embodiment, the calculation portion 16c may calculate the control constant Ns by dividing the specific movement velocity Vs by the specific drive frequency Fs. In this case, the control portion 16d may control the drive frequency F so that the drive frequency F comes close to a value obtained by dividing the movement velocity V of the fluid ejection tube 20 by the control constant Ns.

### Modification Example 3

In the above-mentioned embodiment, the control device 16 may calculate the movement velocity V of the fluid ejection tube 20 on the basis of an image or a moving image which is captured by a camera provided to the handpiece 14 or a camera provided at a position other than the handpiece 14. In addition, in the above-mentioned embodiment, a camera, a sensor or the like that detects the movement (velocity) of an affected part may be provided, and the control device 16 may calculate a relative velocity between an affected part and the fluid ejection tube 20.

### Modification Example 4

In the above-mentioned embodiment, the pulsation generation portion 22 may be a mechanism that generates air bubbles by irradiating a fluid with a laser and generating pulsation due to the air bubbles. In this case, an optical fiber cable for perform irradiation with a laser may be connected to the pulsation generation portion 22. In addition, the pulsation generation portion 22 may be a mechanism that generates air bubbles using an electric heater and generates pulsation.

### Modification Example 5

In the above-mentioned embodiment, the release of the specific drive frequency Fs and the specific movement velocity Vs which are set may be performed when an operator stops the depression of the ejection switch 18, or when an operator depresses the information acquisition switch 26 again. In addition, a button for releasing the specific drive frequency Fs and the specific movement velocity Vs which are set may be separately provided.

### Modification Example 6

The controls performed in the above-mentioned first embodiment to the fifth embodiment may be appropriately combined.

### Modification Example 7

A portion of functions realized by software in the above-mentioned embodiment may be realized by hardware, or a portion of functions realized by hardware may be realized by software.

The invention is not limited to the aforementioned embodiments, examples, and modification examples of this specification, and can be implemented by various configurations without the gist of the invention. For example, technical features in the embodiments, examples, and modification examples which correspond to the technical features in the respective aspects described in the summary of the invention can be appropriately replaced or combined in order to solve some or all of the aforementioned problems, or to achieve some or all of the aforementioned effects.

What is claimed is:

1. A medical device that ejects a fluid, comprising:
a fluid ejection portion that includes an ejection tube having an opening for ejecting the fluid and a pulsation generation portion, communicating with the ejection tube, which generates pulsation to the fluid;
a control portion that controls a frequency of the pulsation by controlling the pulsation generation portion;
a measurement portion that measures a movement velocity of the fluid ejection portion;
a receiving portion that receives an instruction for setting a specific frequency and a specific movement velocity from a user;
a setting portion that sets the specific frequency and the specific movement velocity on the basis of the instruction from a user; and
a calculation portion that calculates a control constant using the specific frequency and the specific movement velocity,
wherein the control portion controls the frequency in accordance with the movement velocity so that a value which is calculated by the same method as a method of calculating the control constant using the frequency and the movement velocity falls within a predetermined range including the control constant.

2. The medical device according to claim 1, wherein the setting portion sets the frequency at a timing when the setting instruction is received, to the specific frequency, and sets the movement velocity at a timing when the setting instruction is received, to the specific movement velocity.

3. The medical device according to claim 1, wherein the calculation portion calculates the control constant by dividing the specific frequency by the specific movement velocity, and
the control portion controls the frequency so that a value of the frequency comes close to a value obtained by multiplying the control constant and the movement velocity together.

4. The medical device according to claim 1, wherein the control portion controls the frequency so that when the movement velocity is larger than a predetermined upper limit threshold, the frequency has a value smaller than a value of the frequency in a case where the frequency is controlled using the control constant.

5. The medical device according to claim 1, wherein the control portion controls the frequency so that when the movement velocity is less than a predetermined lower limit threshold, the frequency has a value smaller than a value of the frequency in a case where the frequency is controlled using the control constant.

6. The medical device according to claim 1, wherein the control portion performs control so that an output of the pulsation generation portion is reduced when the movement velocity is larger than a predetermined upper limit threshold.

7. The medical device according to claim 1, wherein the control portion performs control so that an output of the pulsation generation portion is reduced when the movement velocity is less than a predetermined lower limit threshold.
